(19) United States
(12) Patent Application Publication
(10) Pub. No.: US 2004/0059296 A1
(43) Pub. Date: Mar. 25, 2004

(54) Peripherally inserted peel away introducer cannula assembly with peel away valve

(76) Inventor: Mark W. Godfrey, Ramona, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/251,687

(22) Filed: Sep. 19, 2002

(51) Int. Cl. A61M 5/178

(52) U.S. Cl. 604/164.05; 604/171

(57) ABSTRACT

A peel-away introducer cannula having a sheath tube, cannula hub, and peel-away blood flow reducing valve to prevent backflow of blood is provided. The sheath tube is weakened along opposed axial lines to allow for the sheath tube to be split in two. Similarly, the cannula hub is weakened along opposed axial lines, which align with the opposed axial lines in the sheath tube. The blood flow reducing valve is disposed within the cannula hub, wherein the blood flow reducing valve is also weakened along opposed axial lines, which align with the weakened opposed axial lines of the sheath tube and said cannula hub. Optionally, a splittable handle is attached to the hub such that the handle may be split and drawn apart to split the sheath tube, blood flow reducing valve, and hub along the weakened axial lines. Methods of introducing a vascular device are similarly provided.

### BACKGROUND OF THE INVENTION

1. Field of the Invention

The invention relates generally to a peripherally inserted peel away introducer cannula assembly having a peel away valve to reduce or eliminate blood backflow and methods of using the introducer cannula assembly. The cannula assembly of the invention is useful for accessing an individual's venous system for the placement of indwelling catheters while preventing or reducing blood backflow.

2. Description of the Related Art

As described in U.S. Pat. No. 5,250,033, introducer cannulas are commonly used in various medical procedures where a catheter is transcutaneously introduced to an interior body lumen or cavity, such as a blood vessel or a hollow body organ. Generally, the introducer cannula includes a thin-walled sheath tube tightly fitting over a needle or dilator. The needle and introducer are introduced together into a patient's vascular system. Upon successful insertion the needle is removed, leaving a relatively large diameter working channel defined by the axial lumen of the sheath.

The introducer cannula includes a thin-walled sheath tube which is introduced through a previously formed needle penetration together with an internal dilator, stylet or obturator. Advantageously, the dilator, stylet or obturator has a tapered distal end which extends from the sheath and dilates the previously formed hole as the sheath is advanced. After the combination of the sheath and dilator/stylet/obturator has been introduced, the dilator/stylet/obturator is removed, leaving a relatively large diameter working channel defined by the axial lumen of the sheath.

Various improvements in the design of such introducers have been proposed. Of particular interest are peel-away introducer sheaths wherein the sheath is weakened along opposed axial lines to facilitate splitting when the proximal end of the sheath is pulled apart. Peel-away introducers are described in U.S. Pat. Nos. 5,098,392; 4,983,168; 4,596,559; 4,412,832; and Re 31,855. Such peel-away cannulas are useful when it is desired to remove the cannula from around a catheter or other device which remains in place in the transcutaneous penetration. In particular, the cannula may be withdrawn and split or pulled apart to facilitate removal. Notably, without the ability to peel away the introducer cannula, it would be impossible to remove the cannula over an enlarged proximal hub or housing on the catheter or other device which remains in place.

Another improvement includes the inclusion of a gas or liquid sealing valve on the proximal end of the sheath to permit the exchange of working catheters while the introducer sheath remains in place. The valve serves to minimize gas or liquid loss during an interventional procedure. Peel-away introducers have been proposed which include a gas or liquid sealing valve to permit catheter exchange through the sheath and, after placement of a final catheter, withdrawal and removal of the sheath while the final catheter remains in place.

However, the vast majority of introducer devices on the market today do not address the problems associated with blood backflow during the introduction of a catheter into a patient's venous system. Since the early 1990's, infection by blood-borne pathogens has become a very serious concern to healthcare workers and patients alike. HIV/AIDS and various forms of hepatitis have caused a dramatic shift in the way medical devices are constructed. The elimination of needle sticks and blood contact have been the primary design changes. Many devices are currently marketed as “safety” devices but most fall short of the end goal of preventing the transmission of blood-borne pathogens.

There is a need for introducer devices which prevent backflow of blood from the proximal end of the device. In recent years, concern over bloodborne pathogens has been on the rise. New federal laws have been passed which address both the issue of needle sticks and exposure to bloodborne pathogens such as hepatitis B, hepatitis C, and HIV. Current introducer devices fail to adequately protect healthcare workers and patients from these dangers. Moreover, the only way to protect healthcare workers from potentially deadly bloodborne pathogens is to require them to wear protective clothing such as gloves, masks and gowns, and to use universal precautions. Namely, healthcare workers must treat all body fluids as infectious and take all precautions in handling anything contaminated, and to treat all blood as infectious.

In the case of intravenous catheters, there is an absence of safety devices on the market which reduce or prevent the risk of exposure to blood-borne pathogens to healthcare workers. It is therefore an object of the present invention to provide a true safety device to address the issue of transmission of blood-borne pathogens during the insertion and use of indwelling catheter devices.

Today, all of the “T” peel or peel-away introducers that are currently in use do not have any way to effectively slow down, reduce, or stop the blood outflow from the proximal end of the peel-away sheath. After the introducer cannula has been placed in a patient's vein and the introducer needle has been removed, blood rushes out of the rear of the peel away cannula until such time that the vascular access device is placed or threaded into the opening created by the cannula. At this point, the loss of blood is reduced by the access device filling the internal diameter of the peel-away cannula. Currently, no introducer cannula devices have been proposed which address the blood loss through the proximal end of the peel-away cannula.

It would be a great boon to healthcare workers and patients alike to provide improved, low cost introducer devices which prevent the transmission of bloodborne pathogens and minimize trauma to the patient.

### SUMMARY OF THE INVENTION

The invention is generally directed to a peripherally inserted peel away introducer cannula assembly having a peel away valve to reduce or eliminate blood backflow and methods of using the introducer cannula assembly. The cannula assembly of the invention is particularly useful for accessing an individual's venous system for the placement of indwelling catheters while preventing or reducing blood backflow.

Accordingly, in one aspect of the invention, a peel-away introducer device is provided. The peel-away introducer includes a sheath tube having a distal end, a proximal end, and a lumen therethrough, wherein the sheath is weakened along a first pair of opposed axial lines to facilitate splitting of said hub into portions and a cannula hub attached to the proximal end of the sheath tube, wherein the hub is weakened along a second pair of opposed axial lines, and wherein the second pair of opposed axial lines align with the first pair of opposed axial lines in the sheath tube to facilitate splitting of the hub and the sheath tube into portions. Advantageously, the peel-away introducer also includes a peel-away bloodflow reducing valve disposed within the cannula hub. The blood flow reducing valve may be weakened along a third pair of opposed axial lines, wherein the third pair of opposed axial lines align with the first and second pair of weakened opposed axial lines of the sheath tube and the cannula hub. Preferably, the peel-away introducer cannula includes a splittable handle or wings attached to the hub and has a circular aperture aligned within the tube lumen. Advantageously, the opposing handles or wings are weakened, pre split or completely separated at locations offset from the weakened axial lines in the sheath tube, cannula hub, and blood flow reducing valve so that the handle may be split and drawn apart to split the sheath tube, cannula hub, and blood flow reducing valve along the weakened axial lines. Preferably, the opposing handles or wings are offset 90 degrees from the weakened axial lines in the sheath tube, cannula hub, and blood flow reducing valve.

In another aspect of the invention, the peel-away blood flow reducing valve is a duckbill valve.

In yet another aspect of the invention, a method for transcutaneously introducing a vascular device through an introducer cannula having a peel-away blood flow reducing valve to reduce blood backflow is contemplated. The method includes providing an introducer cannula comprising a peel-away blood flow reducing valve and retracting a needle back through the cannula and past the peel-away blood flow reducing valve, ultimately completely removing the needle. Advantageously, the needle has been penetrated through a patient's skin and into their vascular system leaving the distal tip of the cannula in the patient's vascular system. The method further includes introducing a vascular device through the introducer to the patient's venous system; withdrawing the introducer cannula over the device which remains in the patient's venous system; and splitting or peeling the introducer cannula, such that the introducer cannula and blood flow reducing valve are peeled apart and then discarded. The vascular device may be a PICC, Midline, Peripheral, diagnostic or any vascular access catheter.

A method for reducing transmission of blood-borne pathogens during the introduction of a medical device into a patient's venous system is similarly provided. The method includes: providing an introducer cannula comprising a peel-away blood flow reducing valve; passing a needle through the cannula and past the peel-away blood flow reducing valve; removing the needle from the cannula, wherein the removal of the needle causes the peel-away blood flow reducing valve to close; and introducing a vascular device through the introducer to the patient's venous system. The vascular device may be a PICC, Midline, Peripheral, therapeutic or diagnostic catheter. The blood borne pathogen may be hepatitis B, hepatitis C, or HIV.

In yet another aspect of the invention, a method of preventing blood loss during the introduction of a vascular device into a patient's venous system is disclosed. The method includes providing a peel-away introducer cannula comprising a peel-away blood flow reducing valve; passing a needle through the cannula and past the peel-away blood flow reducing valve, such that the removal of the needle causes the peel-away blood flow reducing valve to close; and introducing a vascular device through the introducer to the patient's venous system. Advantageously, the blood flow reducing valve is a duckbill valve. Optionally, the method further includes withdrawing the introducer over the device which remains in the patient's venous system; and splitting the introducer cannula, such that the introducer cannula and blood flow reducing valve are removed.

In still another aspect of the invention, a method of reducing the potential for development of a vascular embolism during the placement of an indwelling catheter into the venous system of a patient is disclosed. The method includes: providing an introducer cannula comprising a peel-away blood flow reducing valve; passing a needle through the cannula and past the peel-away blood flow reducing valve, such that the removal of the needle causes the peel-away blood flow reducing valve to close; and introducing a vascular device through the introducer to the patient's venous system. In some aspects of the invention, the method of reducing the potential for the development of a vascular embolism during the placement of an indwelling catheter into the venous system of a patient further includes withdrawing the introducer over the device which remains in the patient's venous system; and splitting the introducer cannula, such that the introducer cannula and blood flow reducing valve are removed.

A peel-away duckbill valve for use in a peel-away introducer assembly is disclosed. The valve includes a body member having an axial fluid passageway and a first end and a second end, wherein the body member is weakened along a pair of opposed axial lines; and at least two resilient lips at the second end of the body member defining an outlet slit therebetween, the axial fluid passageway having a cross section that progressively narrows from the first end to the second end. Preferably, the duckbill valve is made of a resilient material such as a thermoplastic or an elastomer.

In yet another aspect of the invention, a peel-away introducer cannula comprising a peel-away blood flow reducing valve for the introduction of a vascular device into a patient's venous system is provided. The peel-away introducer cannula assembly includes a housing comprising a peel-away blood flow reducing valve disposed therein; a means for passing a needle through the cannula and past the peel-away blood flow reducing valve; a means for introducing the vascular device through said housing to the patient's venous system; a means for withdrawing the housing over the device which remains in the patient's venous system; and a means for splitting the housing such that the housing and peel-away blood flow reducing valve are removed.

In one aspect of the invention, a method of manufacturing a peel-away introducer cannula assembly having a peel-away valve includes the steps of providing a sheath tube having a distal end, a proximal end, and lumen therethrough; weakening the sheath tube along a first pair of axial lines to facilitate splitting of the sheath tube into portions; locating a cannula hub at the proximal end of the sheath tube, wherein the cannula hub is weakened along a second pair of opposed axial lines; aligning the first pair of opposed axial lines of the sheath tube with the second pair of opposed axial lines of the cannula hub; disposing within the cannula hub a peel-away blood flow reducing valve, wherein the peel-away blood flow reducing valve is weakened along a third pair of opposed axial lines to allow for splitting of the valve into portions; aligning the third pair of opposed axial lines with the first and second pair of axial lines of the sheath tube and hub; and connecting a splittable handle to the cannula hub.

In another aspect of the invention, a peel-away introducer device is disclosed. The introducer device has a sheath tube having a distal end, a proximal end, and a lumen therethrough, wherein the sheath tube is weakened along a first pair of opposed axial lines to facilitate splitting of said tube into portions. Also included in the introducer device is a cannula hub attached to the proximal end of the sheath tube, wherein the hub is weakened along a second pair of opposed axial lines, wherein the second pair of opposed axial lines align with the first pair of opposed axial lines in said sheath tube to facilitate splitting of the hub into portions; and a peel-away duckbill valve disposed within the cannula hub, wherein the valve is weakened along a third pair of opposed axial lines, and wherein the third pair of opposed axial lines align with the first and second pair of weakened axial lines of the sheath tube and the cannula hub. The introducer device additionally includes a splittable handle attached to the hub and having a circular aperture aligned within the tube lumen. Advantageously, the handle is weakened at locations aligned with the weakened axial lines in the sheath tube, cannula hub, and said peel-away duckbill valve so that the handle may be split and drawn apart to split the sheath tube, cannula hub, and peel-away duckbill valve along the weakened axial lines.

### BRIEF DESCRIPTION OF THE DRAWINGS

FIG. 1 is partial cut away view of a peel away introducer having a peel-away blood-eliminating valve.

FIG. 2 is a cross sectional view of a cannula hub having a peel-away blood flow reducing valve disposed therein.

FIG. 3A is a front view of one embodiment of a peel-away blood flow reducing valve for use in the present invention.

FIG. 3B is a side view of one embodiment of a peel-away blood flow reducing valve suitable for use in the present invention.

FIG. 3C is a top view of one embodiment of a peel-away blood flow reducing valve suitable for use in the present invention.

FIG. 4 is a cross sectional view of a cannula assembly having a needle disposed therethrough.

### DETAILED DESCRIPTION OF THE INVENTION

The invention relates to peel away valve assemblies including pressure responsive valves such as a duckbill valve and normally closed valves. In particular, the invention relates to a peel-away introducer cannula assembly having a peel away valve disposed within the cannula to prevent fluid backflow.

Embodiments of the invention will now be described with reference to the accompanying Figures, wherein like numerals refer to like elements throughout. The terminology used in the description presented herein is not intended to be interpreted in any limited or restrictive manner, simply because it is being utilized in conjunction with a detailed description of certain specific embodiments of the invention. Furthermore, embodiments of the invention may include several novel features, no single one of which is solely responsible for its desirable attributes or which is essential to practicing the inventions herein described.

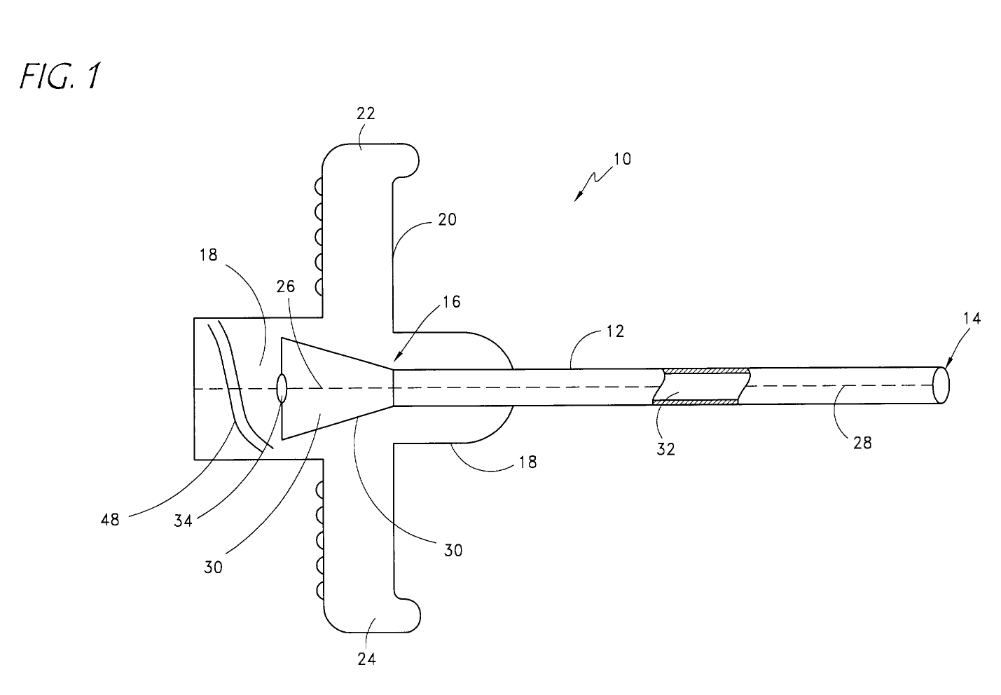

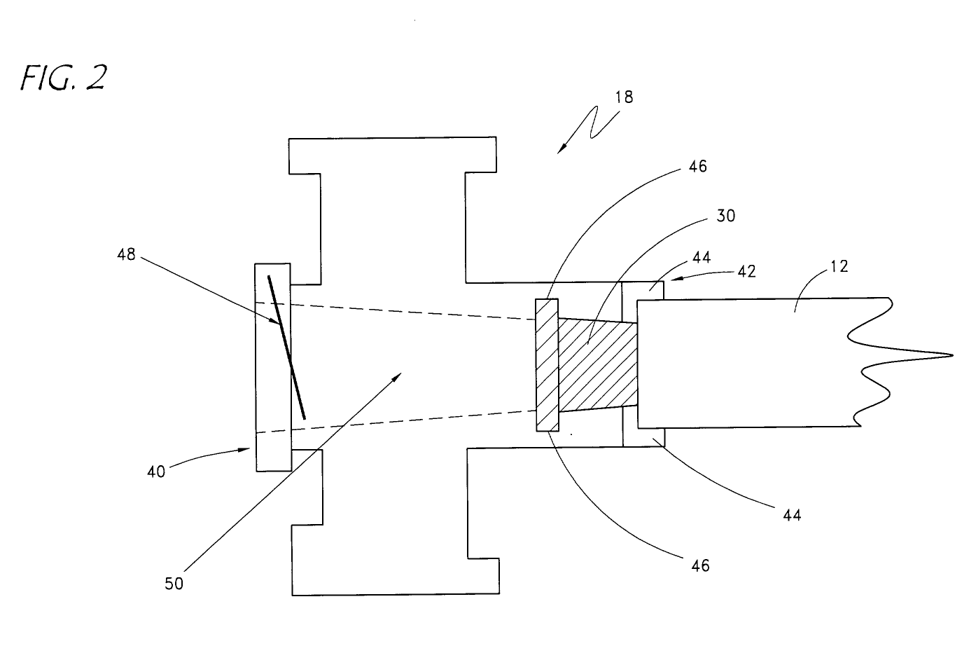

FIG. 1 is a partial cut-away view of one embodiment of the peel-away introducer cannula assembly 10 of the invention. The peel-away introducer cannula assembly 10 comprises a thin-walled sheath tube 12 having a distal end 14 and a proximal end 16. At the proximal end 16 a cannula hub 18 is fixedly attached to the sheath tube 12. The cannula hub 18 includes a handle 20 located generally between the proximal and distal end of the cannula hub 18. The handle 20 includes a first tab 22 and a second tab 24. The cannula hub 18 further comprises a pair of thin or weakened axial grooves or lines 26 extending from the distal end to the proximal end of the cannula hub 18, which coincide with a pair of thin or weekend axial grooves or lines 28 on the sheath tube 12. In preferred embodiments, the lines 26 are diametrically opposed to each other. As used herein, the phrase “thin lines” encompasses any weakened or thin surface region along portions of the housing which are capable of being split when apart along the weakened lines when a force is applied. The thin lines 26 divide the cannula hub 18 into two portions, where each portion is attached to one half of the handle 20. Accordingly, the cannula hub 18 can be pulled apart along the thin lines 26 by breaking the handle 20 apart and pulling outwardly on the first tab 22 and second tab 24. Disposed within the cannula hub 18 at the proximal end 16 of the sheath tube 12, a blood flow reducing valve 30 is secured to an interior portion of the cannula hub 18 as will be described in greater detail with reference to FIG. 2. Attachment of the blood-eliminating valve 30 to the interior portion of the hub 18 is accomplished via conventional means such as chemical or thermal bonding, mechanical compression, or other means such as insert molding. In one embodiment, a retaining mechanism (not illustrated) can be employed to hold the valve 30 in place. The valve 30 is positioned toward the rear of the hub 18 such that the unimpeded opening and closing of the valve. A connector 48 is located at of the cannula hub. The connector can be threaded or non-threaded such as pe device. Advantageously, the connector 48 is designed to mate the for example, a safety needle mechanism.

Still with reference to FIG. 1, the introducer 10 defines an access lumen men 32 extending from the distal end 14 to the proximal end 16 of the sheath rture (not shown) formed in the hub 18, and an axial passage 34 formed d flow reducing valve 30.

The sheath tube 12 is typically an elongated cylindrical tube formed from a ible plastic, such as polyethylene, tetrafluorethylene, fluorinated ethylene imilar material. The tube can be extruded by conventional methods. The also includes a pair of thin or weakened axial grooves or lines 28 extending o the proximal end of the sheath tube. In preferred embodiments, the lines 28 y opposed to each other. The thin lines 28 divide the sheath tube 12 into two s. Thus, like the cannula hub 18, the sheath tube 12 can be pulled apart by dle 20 apart and pulling outwardly on the first tab 22 and second tab 24.

A person of skill in the art will appreciate that the dimensions of the re not an essential feature of the invention. Generally, the sheath tube 12 has ange from about 2 cm to 30 cm. In preferred embodiments, the length of the etween about 5 cm and 20 cm. The internal diameter of the sheath tube 12 is 0.5 mm and 10 mm. The thickness of the wall of the sheath tube 12 is en about 0.01 mm and 0.5 mm.

FIG. 2 is a cross-sectional view of the cannula hub 18 portion of the cer 10. The cannula hub 18 includes a proximal end 40 and a distal end 42. nd 42 of the cannula hub 18, the sheath tube 12 of the introducer is insert with a bioadhesive such as Loctite 4001 or otherwise affixed to the cannula bonded joints 44. Disposed within the interior of the distal end 42 of the , a blood flow reducing valve 30 is provided. The blood flow reducing valve osition within the interior portion of the cannula hub 18 by a pair of locking tabs or collar 46. At the proximal end 40 of the cannula hub 18, a connector 48 is optionally provided. Between the connector 48 and the blood-eliminating valve, a cavity 50 is defined.

Thus far, the construction of the introducer 10 is largely conventional with the exception of the peel-away blood flow reducing valve assembly disposed within the cannula hub 18 and attached to the handle 20 of the cannula hub 18. As described above, the blood flow reducing valve 30 is fixedly attached to the an interior portion of the cannula hub 18. The blood flow reducing valve 30 can be any valve assembly which is ordinarily in a closed position and is capable of being peeled away. Examples of suitable valve assemblies include, without limitation, pressure responsive valves, duckbill valves, and flap valves. As used herein, the term “blood-eliminating valve” includes generally all valves that allow flow in only one direction and enable the insertion of vascular devices.

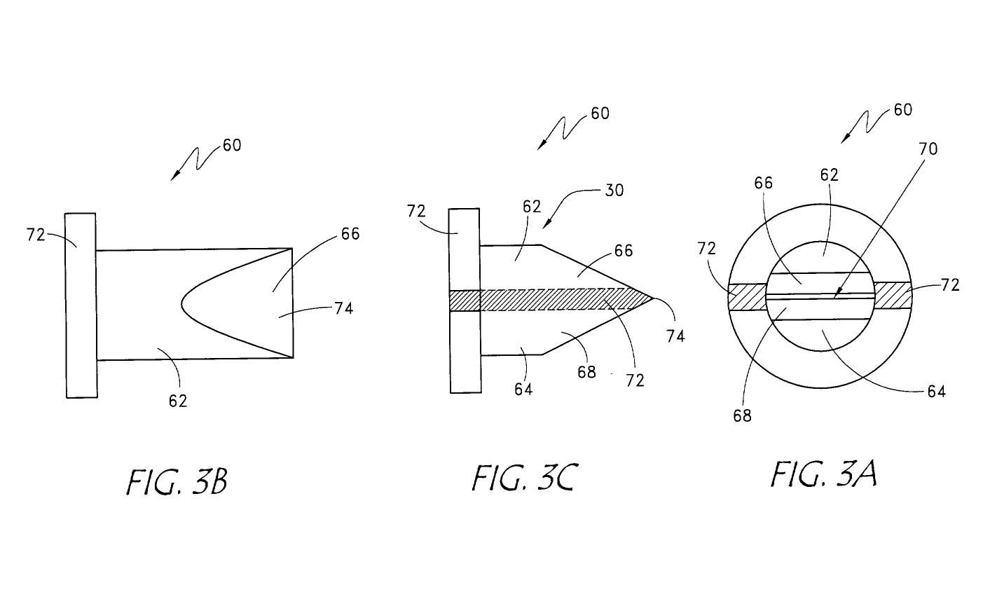

As illustrated in FIGS. 3A, 3B, and 3C, in a preferred embodiment, the blood flow reducing valve 30 is a duckbill valve. In the prior art, a duckbill valve has been typically used as a one-way, on/off flow valve which allows free flow in a first direction while preventing flow through the valve in a second opposite direction. See, e.g. U.S. Pat. No. 4,535,819 (disclosing a duckbill valve assembly). In typical operation, the valve is positioned in line with a fluid conducting conduit. The outer perimeter of a first cylindrical end of the valve is sealed with the inner wall of the conduit. The body of the valve tapers down to a pair of resilient lips at a second end of the valve which form an outlet slit.

FIGS. 3A, 3B, and 3C illustrate one embodiment of the blood flow reducing valve 30. FIG. 3A is a front view of a duckbill valve assembly suitable for use in the invention. FIG. 3B is a side view of a duckbill valve assembly and FIG. 3C is a top view of a duckbill valve assembly suitable for use in the invention. The valve assembly comprises a body 60 having two body members 62 and 64 (See FIG. 3C). The body members 62 and 64 smoothly taper down to form a pair of resilient lips 66 and 68. Between the pair of resilient lips 66 and 68, an outlet slit 70 is formed. Duckbill valves of this general type are commercially available from such sources as Vemay Laboratories, Inc. and Da/Pro Rubber Inc. of Tulsa, Okla. The duckbill valve is preferably made of a resilient material, such as a thermoplastic or an elastomer. Silicone valves are particularly preferred for their ease of splitting. In particularly preferred embodiments, the valves are constructed of latex free materials.

The blood flow reducing valve 30 also includes a pair of thin or weakened axial grooves or lines 72 which extend from the proximal to the distal end of the valve body 60. In preferred embodiments, the lines 72 are diametrically opposed to each other. The thin lines 72 divide the blood flow reducing valve 30 into two portions. Thus, like the cannula hub 18 and the sheath tube 12, the blood flow reducing valve 30 can be pulled apart by breaking the handle 20 of the cannula hub 18 apart and pulling outwardly on the first tab 22 and second tab 24, thereby separating the blood flow reducing valve 30 into two pieces and facilitating removal of the entire peel-away introducer.

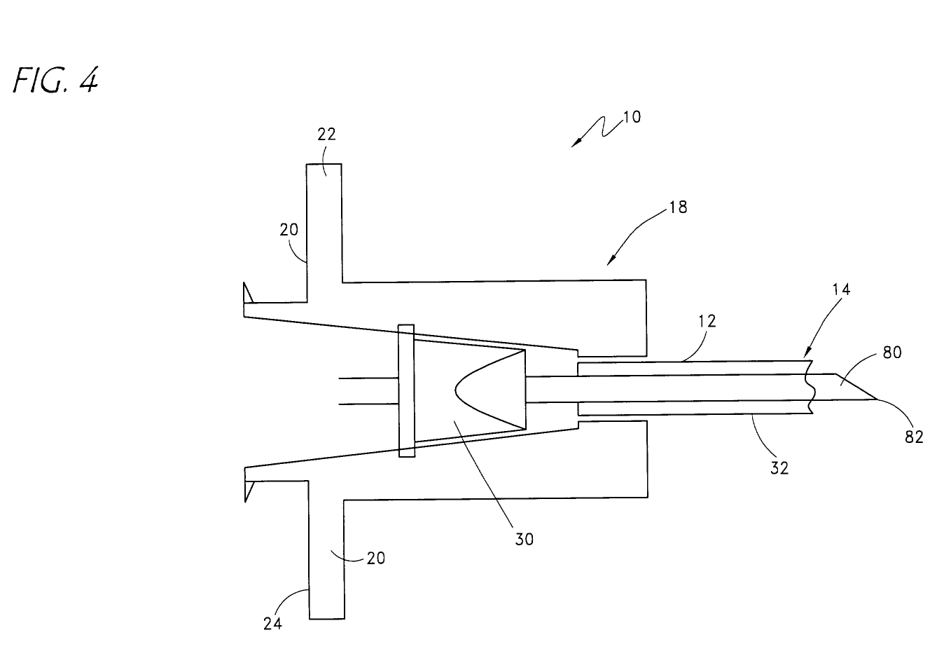

With reference to FIGS. 3B and 3C, the body 60 has a first end 72 and a second end 74. As a needle, dilator, stylet, or obturator is passed through the introducer 10, the needle enters the first end 72 and passes through the second end 74 of the blood flow reducing valve 30. In the course of passing through the body of the valve assembly 30, the needle, dilator, stylet or obturator forces the two resilient lips 66 and 68 outward and enlarges the outlet slit 70, thereby creating a channel for the needle to pass through, into the sheath tube 12 of the introducer 10, and ultimately into the venous system of a patient. Upon removal of the needle, dilator, stylet or obturator, the resilient lips 66 and 68 return to their normally closed position and force the outlet slit 70 closed, thereby preventing any backflow of blood during the insertion of the introducer device. The insertion of the introducer and role of the peel-away valve assembly will be described in greater detail with reference to FIG. 4.

As illustrated in FIG. 4, in operation, the peel-away introducer 10 is transcutaneously introduced to a desired body lumen or cavity, typically a blood vessel, hollow body organ, peritoneal space, or the like, by conventional techniques. FIG. 4 is a cross-sectional view of an introducer cannula 10 with a stylet, obturator, or needle 80 placed through the cannula hub 18 of the introducer 10 and is passed through the blood flow reducing valve 30. In preferred embodiments, the blood flow reducing valve 30 is a duckbill valve. As the needle, dilator, stylet, obturator, or vascular device is passed through the body members 62 and 64 of the blood flow reducing valve 30, a force is applied to the resilient lips 66 and 68, opening the outlet slit 70, and provides a passageway for the needle, stylet, obturator, or vascular device to travel through the outlet slit 70, past the sheath tube 12, and into a patient's venous system. Once the needle, stylet, or obturator 80 is removed from the cannula introducer 10, the pressure on the second end 74 of the valve exceeds the pressure on the first end 72, and the pair of resilient lips 66, 68 are forced against each other, closing the outlet slit 70 and preventing, or at least reducing, the flow of blood through the resilient lips 66, 68 and out through the body members 62, 64 of the blood flow reducing valve 30.

Typically, a patient's venous pressure is approximately 30 mmHg. In preferred embodiments, the closing of the pair of resilient lips 66, 68 of the duckbill valve is facilitated by the venous pressure on the second end 74 of the valve imparted by the flow of blood back from the venous system of the patient outward towards the resilient lips 66, 68. Upon retraction of the needle through the valve 30, the patient's venous pressure causes blood to flow from the patient's venous system out through the distal end of the cannula 10. Once the needle is pulled through the valve 30, the blood following down the cannula 10 puts pressure on the outer lips 66, 68 of the duckbill, forcing the outlet slit 70 to close to ensure that the lips are forced into a closed position. In so doing, the risk of additional blood loss is eliminated.

The introducer 10 is initially placed over a dilator, stylet, obturator, or needle 80 which extends through the access lumen 32 and includes a tapered distal end 82 extending out of the distal end 14 of the sheath tube 12. Using the tapered end 82 of the stylet, obturator, or needle as a dilator, the introducer 10 can be passed into a target lumen, particularly a patient's vascular system, through a formed or previously formed hole. Once in place, the dilator, stylet, obturator, or needle 80 is retracted by pulling it through the sheath tube 12, past the blood flow reducing valve 30, and out the cannula hub 18. As the dilator, stylet, obturator or needle 80 is removed, the blood flow reducing valve 30 closes and cuts off blood flow, thereby preventing blood from exiting out the cannula hub 18. The access lumen 32 of the introducer 10 is still available for use in subsequent procedures such as the placement of an indwelling catheter, diagnostic catheter, or other device into the vascular system of a patient. Suitable indwelling catheters include, without limitation, Peripherally Inserted Central catheters (“PICCs”), Midline (“Mid”) catheters, Peripheral diagnostic and therapeutic catheters. Examples of diagnostic catheters include, without limitation, those catheters used in angiography; advanced sensor catheters for measuring blood oxygen, venous oxygen, and cardiac output; and diagnostic imaging catheters. Examples of therapeutic catheters include, without limitation, atherectomy, angioplasty, RF ablation, etc.

In preferred embodiments, upon removal of the dilator, stylet, obturator, or needle 80, a vascular device such as an indwelling catheter is threaded through the proximal end 86 of the introducer 10. The threading continues into a patient's venous system. Once an indwelling catheter is inserted into a patient, the peel-away introducer 10 is removed from the patient's vein, moved up the access device a few inches, and is then peeled apart. The peeling apart of the introducer 10 along the weakened lines 26, 28 of the cannula hub 18 and sheath tube 12, respectively, is accomplished by pulling the first tab 22 and second tab 24 of the handle 20 in opposite directions, thereby splitting the cannula hub 18 and sheath tube 12 along the thin axial lines 26, 28. The blood flow reducing valve 30 is similarly peeled into two portions along the axial weakened lines 72 (not illustrated), which remain attached to the two portions of the cannula hub 18. Upon completion of the peel-away process, the introducer 10 can be disposed of.

The invention provides a method of reducing the transmission of bloodborne infections from patients to healthcare workers during the introduction of a catheter into a patient's vascular system. A peel-away introducer comprising a peel-away blood flow reducing valve is introduced into the venous system of a patient using a needle, dilator, stylet, or obturator. As the needle, dilator, stylet, or obturator is retracted, the peel-away blood flow reducing valve closes and prevents blood from flowing out of the proximal end of the introducer assembly 10. Because blood is blocked as the peel-away blood flow reducing valve is closed, the healthcare worker inserting the introducer and any subsequent vascular devices through the introducer is protected from exposure to patient blood. Accordingly, the risk of transmission of blood-borne pathogens such as HIV, various strains of hepatitis, etc. is reduced or eliminated.

In another embodiment, a method of reducing patient blood loss during the placement of a vascular device into a patient's venous system is provided. Typically, when vascular devices such as indwelling catheters or diagnostic catheters are placed in a patient's venous system, blood will seep out of the proximal end of the introducer. By employing the introducer assembly of the present invention, blood loss is minimized. The blood flow reducing valve prevents the backflow of blood out of the introducer device. Accordingly, the blood stays in the patient and does not flow unabated out of the introducer device.

A method of manufacturing a peel-away introducer having a peel-away blood flow reducing valve is likewise provided. The method includes providing a sheath tube having a distal end, a proximal end, and lumen therethrough. The sheath tube is weakened along a first pair of opposed axial lines to facilitate splitting of the sheath tube into portions. A cannula hub is attached to the proximal end of the sheath tube. The hub is weakened along a second pair of opposed axial lines. The opposed axial lines of the hub are aligned with the first pair of opposed axial lines of the sheath tube. A peel-away blood flow reducing valve is located within the cannula hub and attached to an interior portion of the cannula hub. The peel-away blood flow reducing valve is weakened along a third pair of opposed axial lines to allow for splitting of the valve into portions. The third pair of opposed axial lines are aligned with the first and second pair of axial lines of the sheath tube and hub. A splittable handle is attached to the cannula hub.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

What is claimed is:

1. A peel-away introducer device comprising:
a sheath tube having a distal end, a proximal end, and a lumen therethrough, wherein said sheath is weakened along a first pair of opposed axial lines to facilitate splitting of said tube into portions;
a cannula hub attached to the proximal end of said sheath tube, wherein said hub is weakened along a second pair of opposed axial lines, wherein said second pair of opposed axial lines align with the first pair of opposed axial lines in said sheath tube to facilitate splitting of said hub and said sheath tube into portions; and
a peel-away blood flow reducing valve disposed within said cannula hub, wherein said blood flow reducing valve is weakened along a third pair of opposed axial lines, wherein said third pair of opposed axial lines align with the first and second pair of weakened opposed axial lines of said sheath tube and said cannula hub.

2. The peel-away introducer of claim 1, wherein said blood flow reducing valve is a duckbill valve.

3. The peel-away introducer of claim 1, wherein said hub comprises a splittable handle attached to said hub and having a circular aperture aligned within the tube lumen, said handle being weakened at locations aligned with the weakened axial lines in the sheath tube, cannula hub, and blood flow reducing valve so that the handle may be split and drawn apart to split the sheath tube, cannula hub, and blood flow reducing valve along said weakened axial lines

4. A method for transcutaneously introducing a vascular device through an introducer cannula having a peel-away blood flow reducing valve to reduce blood backflow, comprising:
providing an introducer cannula comprising a peel-away blood flow reducing valve and a needle disposed therethrough;
retracting said needle through said peel-away blood flow reducing valve and past said cannula to withdraw said needle;
introducing a vascular device through said introducer to the patient's venous system;
withdrawing said introducer over the device which remains in the patient's venous system;
splitting the introducer cannula, such that the introducer cannula and blood flow reducing valve are removed from said vascular device.

5. The method of claim 4, wherein said device is selected from the group consisting of a PICC, Midline, and Peripheral catheter.

6. The method of claim 4, wherein said device is a therapeutic catheter.

7. The method of claim 4, wherein said device is a diagnostic catheter.

8. The method of claim 4, wherein said blood flow reducing valve is a duckbill valve.

9. The method of claim 4, wherein said blood flow reducing valve is a one way valve.

10. The method of claim 4, wherein said blood flow reducing valve is a pressure responsive valve.

11. A method for reducing transmission of bloodborne pathogens during the introduction of a medical device into a patient's venous system comprising:
providing an introducer cannula comprising a peel-away blood flow reducing valve and a needle disposed therethrough;
retracting said needle through said peel-away blood flow reducing valve and past said cannula to withdraw said needle;
removing said needle from said cannula, wherein the removal of said needle causes said peel-away blood flow reducing valve to close; and
introducing a vascular device through said introducer to the patient's venous system.

12. The method of claim 11, wherein the closing of said peel-away blood flow reducing valve is facilitated by the pressure imparted by said patient's venous system.

13. The method of claim 11, wherein said vascular device is selected from the group consisting of a PICC, Midline, and Peripheral dwelling catheter.

14. The method of claim 11, wherein said device is a therapeutic catheter.

15. The method of claim 11, wherein said device is a diagnostic catheter.

16. The method of claim 11, wherein said blood borne pathogen is selected from the group consisting of hepatitis B, hepatitis C, and HIV.

17. A method of preventing blood loss during the introduction of a vascular device into a patient's venous system comprising:
providing a peel-away introducer cannula comprising a peel-away blood flow reducing valve and a needle disposed therethrough;
retracting said needle through said peel-away blood flow reducing valve and past said cannula to withdraw said needle;
removing said needle from said cannula, wherein the removal of said needle causes said peel-away blood flow reducing valve to close; and
introducing a vascular device through said introducer to the patient's venous system.

18. The method of claim 17, wherein said blood flow reducing valve is a duckbill valve.

19. The method of claim 17, wherein said blood flow reducing valve is normally closed one-way valve.

20. The method of claim 17, further comprising withdrawing said introducer over the device which remains in the patient's venous system; and
splitting the introducer cannula, such that the introducer cannula and blood flow reducing valve are removed.

21. The method of claim 17, wherein said vascular device is a PICC, Midline, or Peripheral catheter.

22. The method of claim 17, wherein said vascular device is a diagnostic catheter.

23. A peel-away duckbill valve for use in a peel-away introducer assembly comprising:
a body member having an axial fluid passageway and a first end and a second end, wherein said body member is weakened along a pair of opposed axial lines; and
at least two resilient lips at the second end of the body member defining an outlet slit therebetween, the axial fluid passageway having a cross section that progressively narrows from the first end to the second end.

24. The peel-away duckbill valve of claim 23, wherein said valve is made of a resilient material.

25. The peel-away duckbill valve of claim 24, wherein said resilient material is a thermoplastic or an elastomer.

26. The peel-away duckbill valve of claim 25, wherein said resilient material is latex-free.

27. A peel-away introducer cannula comprising a peel-away blood flow reducing valve for the introduction of a vascular device into a patient's venous system comprising:
a housing comprising a peel-away blood flow reducing valve disposed therein;
means for passing a needle through said cannula and past said peel-away blood flow reducing valve through tissue to access a patient's venous system;
means for retracting a needle through said peel-away blood flow reducing valve and past said cannula to withdraw said needle;
means for introducing said vascular device through said housing to the patient's venous system;
a means for withdrawing said housing over said device which remains in the patient's venous system; and
a means for splitting the housing such that the housing and peel-away blood flow reducing valve are removed.

28. The introducer cannula of claim 27, wherein said peel-away blood flow reducing valve is a duckbill valve.

29. The introducer cannula of claim 27, wherein said vascular device is selected from the group consisting of a PICC, Midline, and Peripheral catheter.

30. A method of manufacturing a peel-away introducer cannula assembly having a peel-away valve comprising:
providing a sheath tube having a distal end, a proximal end, and lumen therethrough;
weakening said sheath tube along a first pair of axial lines to facilitate splitting of the sheath tube into portions;
locating a cannula hub at the proximal end of said sheath tube, wherein said cannula hub is weakened along a second pair of opposed axial lines;
aligning said first pair of opposed axial lines of said sheath tube with said second pair of opposed axial lines of said cannula hub;
disposing within said cannula hub a peel-away blood flow reducing valve, wherein said peel-away blood flow reducing valve is weakened along a third pair of opposed axial lines to allow for splitting of the valve into portions;
aligning the third pair of opposed axial lines with the first and second pair of axial lines of the sheath tube and hub; and
connecting a splittable handle to the cannula hub.

31. The method of claim 30, wherein said valve is a duckbill valve.

32. A peel-away introducer device comprising:
a sheath tube having a distal end, a proximal end, and a lumen therethrough, wherein said sheath tube is weakened along a first pair of opposed axial lines to facilitate splitting of said tube into portions;
a cannula hub attached to the proximal end of said sheath tube, wherein said hub is weakened along a second pair of opposed axial lines, wherein said second pair of opposed axial lines align with the first pair of opposed axial lines in said sheath tube to facilitate splitting of the hub into portions; and
a normally closed peel-away blood flow reducing valve disposed within said cannula hub, wherein said valve is weakened along a third pair of opposed axial lines, wherein said third pair of opposed axial lines align with the first and second pair of weakened axial lines of said sheath tube and said cannula hub; and
a splittable handle attached to said hub and having a circular aperture aligned within the tube lumen, said handle being weakened at locations aligned with the weakened axial lines in the sheath tube, cannula hub, and said peel-away valve so that the handle may be split and drawn apart to split the sheath tube, cannula hub, and peel-away valve along said weakened axial lines.

33. The peel-away introducer device of claim 32, wherein said normally closed peel-away valve is a duckbill valve.
